(19) United States
(12) Patent Application Publication
(10) Pub. No.: US 2003/0154506 A1
(43) Pub. Date: Aug. 14, 2003

(54) Process of generating stem cells equivalent to human embryonic stem cells

(76) Inventor: Gin Wu, Corte Madera, CA (US)

Correspondence Address: ROYAL W. CRAIG, A PROFESSIONAL CORPORATION, SUITE 153, 10 NORTH CALVERT STREET, BALTIMORE, MD, 21202, US

(21) Appl. No.: 10/353,771

(22) Filed: Jan. 28, 2003

Related U.S. Application Data
(60) Provisional application No. 60/352,408, filed on Jan. 29, 2002.

(51) Int. Cl. C12N 15/00

(52) U.S. Cl. 800/21

(57) ABSTRACT

A method for the creation of stem cells substantially equivalent to the embryonic stem cells or embryonic germ cells, but without legal or ethical complications. The method is illustrated in the context of the creation of a totipoent cell equivalent to a human fertilized egg by total asexual process. The present method uses somatic cell nuclear transfer (SCNT) technology by which the nucleus of an egg from a mammalian, other than human, was removed with care to avoid significant disruption of the egg plasma. Then a human somatic cell (any body cell other than germ cells) is arranged side by side with the de-nucleated egg. The de-nucleated egg and the human somatic cell are fused by chemical induced cell fusion, and mitochondria are transferred from the nucleus donor to the de-nucleated animal egg. After the fusion, the newly formed chimeric cell may be properly stimulated to start development, or stored cryogenically for later use. The method of the present invention will generate stem cells equivalent to the human embryonic stem cell or human embryonic germ cell without the controversy associated with the destruction of human embryos or the concern with human cloning.

### CROSS-REFERENCE TO RELATED APPLICATION(S)

The present application derives priority from U.S. provisional application serial No. 06/352,408 filed 29 Jan. 2002.

### BACKGROUND OF THE INVENTION

1. Field of the Invention

The present invention relates to the field of generating human embryonic stem cells, specifically to the creation of chimeric embryo asexually and production of stem cells equivalent to human embryonic stem cells without legal and ethical complications.

2. Description of the Background

With current extraordinarily advanced medicine, biotechnology, medical diagnostic equipment and surgical techniques, physiological diseases such as heart attack, kidney failure, diabetes, Parkinson's and Alzheimer's diseases are less devastating, yet there is still no cure for these diseases. Nevertheless, the recent discovery of the multi-potent stem cells in modern developmental biology research had provided unprecedented potentials of finding the cure for these diseases.

Aside from certain successful experiments with mice, recently scientists at Düsseldorf, Germany's Heinrich-Heine-University said they have succeeded for the first in treating a heart attack patient using his own stem cells. After a massive heart attack, the patient had large parts of muscle damage on the left chamber of his heart. The physicians isolated stem cells from patient's bone marrow four days after the heart attack. The stem cells were injected into the patient's affected artery and eventually reached the heart muscle. The patient's heart showed clear improvement over a 10-week follow-up examination. In light of the great promise, clinical researchers are placing a marked emphasis on stem cell research

Stem cells include embryonic stem (ES) cells, embryonic germ (EG) cells, fetal stem cells, and adult stem cells. Stem cells are capable of multiplying for indefinite periods in a properly formulated culture environment. Stem cells also have the potential to further differentiate into many different specialized cells. The process of stem cell formation can be illustrated by following the embryonic developmental process of high-class animal or mammalian. Human development begins when a sperm fertilizes an egg and creates a single cell—the fertilized egg that has the potential to develop into an entire organism. In the stem cell context, the fertilized egg is called a “totipotent” cell. Likewise, the totipotent cell has the potential to develop into any type of cell in a normal animal. Within the first hour of fertilization, the fertilized egg will divide into two identical cells. Either one of these two cells still has the full potential to develop into an entire organism. As matter of a fact, during the next several cell divisions each of all the resulting cells also have the full potential to develop into an entire organism—identical twins, if they were separated and implanted into uterus. These cells are all totipotent cells. However, this separation can't be done indefinitely due to the significant loss of cell mass by the separation. Approximately four days without interruption after fertilization and after several cycles of cell division, these totipotent cells begin to differentiate and form a hollow sphere of cells, called blastocyst.

The blastocyst has an outer layer of cells formed the hollow sphere, a cluster of cells are inside the hollow sphere called the inner cell mass. In the uterus, the outer layer of cells will continue to develop into the placenta and other supporting tissues needed for fetal development in the uterus. The cells of inner cell mass cells will continue to develop into virtually all of the tissues of the human body. However without the support from the outer layer cells, the inner cell mass alone can not form the placenta and will not survive to become a live fetus. These inner cell mass cells are potential to give rise to many types of cells, but not type of cells required for a normal organism. These cells are called “pluripotent cells”. Each of these cells alone has lost the potential to develop into a normal fetus. Therefore, they are not totipotent cells. During development, these pluripotent stem cells will undergo further differentiation into next level of less potent stem cells whose potential to develop into different types of tissues are confined in less variety of cell types.

Another of the commonly studied stem cells is the blood stem cell. It has the potential to give rise to all kinds of blood cells, including red blood cells, white blood cells and platelets. These are more specialized but less potent stem cells, and they are called “multipotent cells.”

At present, totipotent and pluripotent stem cells are only discovered in the early stage embryo. Multipotent stem cells are found not only in later stage of embryos but also found in children and adults. For example, blood stem cells are the best-studied type of stem cells. Blood stem cells can be isolated from bone marrow of both children and adult, as well as very small numbers in the circulating blood stream. These blood stem cells are taking the responsibility of replenishing our blood and immune cells. Other stem cells are considered having the similar function to the body.

From research view, pluripotent stem cells are more valuable to researchers than the totipotent stem cells. This is because totipotent cells have a very broad potential, yet they are difficult to employ in a controlled study. Pluripotent stem cells are more specialized and it is much easier to conduct controlled studies. The study of pluripotent stem cells could offer the detail of complex events that occur during human development and the control of cell differentiation. It will help researchers to identify all the regulatory factors or growth factors essential to the cellular decision making for cell differentiation and tissue development but no longer exist in later time in the life. It also can reveal the genetic control mechanisms which haven been identified or understood in today's medical researches. These mechanisms are extremely important for certain very serious medical complications, such as the aberrant cell division, multiplication or differentiation of cancer cells, birth defects, trauma repairing and chemical induced abnormalities. By understanding these mechanisms, it will dramatically change and improve the current approaches to drug design and development. Furthermore, pluripotent cells are more suitable testing targets for the drugs that affect the cell growth and differentiation.

Furthermore, human pluripotent stem cells give the hope of so-called “cell therapy”. For many diseases that result from destruction of the tissue or disruption of part of cellular functions organ transplantation often become the final choice. Unfortunately, most organ transplant technology is still at high failure rate and the supply of organs is always short. Human pluripotent stem cells can be stimulated to differentiate into specialized cells and used for tissue repairing, therefore reducing the need for organ transplant. This also offers possible treatment to certain diseases generally considered to be untreatable, such as Alzheimer's, Parkinson's, spinal injury, chronic heart disease, stroke, rheumatoid arthritis and osteoarthritis.

Despite the bright future prospects of stem cells in medical application, current medical knowledge is very limited. Moreover, the research community, religious groups and ethnic groups across the country are at odds over the moral and ethical implications involved in destroying a human embryo, and whether the greater good is enough to justify this act. Certain religious communities believe that life starts at the moment of conception, as long as it is genetically human with potential to develop into a human. Some groups believe that the early embryos have no moral protections as the later stage fetus that is much closer to the human during the development. Some groups insist that only stillborns or natural miscarriages should be used. Unfortunately, these often involve some developmental or genetic abnormality and are poor candidates for research.

Despite the public debate, two human puripotent cell lines with partial characterization have been created, both in November of 1998. One line was created by directly isolating the inner cell mass from the blastocysts that were the left over of excess early stage embryos made for the infertility treatment through In Vitro Fertilization. See, Michael Shamblott, et al, Derivation of pluripotent stem cells from cultured human primordial germ cells. PNAS, 95: 13726-13731, November 1998. Then these cells were continuously cultured into pluripotent stem cells. This stem cell line was created from the very early embryonic stage (4˜5 days); it was categorized as human embryonic stem cell.

The second line was created by isolating pluripotent stem cells from fetal tissue obtained from terminated pregnancies. See, James Thomson, et al, Embryonic stem cell lines derived from human blastocysts. Science, 282: 1145-1147, Nov. 6, 1998. In order to distinguish the differences in origin, this stem cell line was called human embryonic germ cells. Since the embryonic germ cells are further down stream in development (5˜9 weeks), the range of the potency as a stem cell is relatively limited to the embryonic stem cell.

There is a third possible way to create pluripotent stem cells that may avoid many of the moral and ethical problems. This is by using somatic cell nuclear transfer technology (SCNT). By fusing a somatic cell (any cell in the body other than sperm and egg) with a de-nucleated egg, the egg will be stimulated to start developing as a fertilized egg. A mammalian egg (other than human) can be used and hence there is much less controversy. The resulting fertilized egg-like cell has the full capacity of a totipotent cell and its descendants are considered fully compatible with the embryo and embryonic stem cells. In SCNT technology, virtually any somatic cell from any individual could be used as nucleus donor. Therefore, for a chronic disease patient there is a possibility of customized cloning of embryonic stem cells. This process will eliminate the potential problem of tissue immuno-incompatibility or rejection for some patients associated embryonic stem cell treatment originated from other individual. SCNT technology has been applied to the animal cloning, and it has been discussed in the context of human somatic cell nuclear transfer. General descriptions of SCNT technology can be found at Philip Cohen, Organs without Donors, New Scientist, July 1998, pp. 4-5; Peter Moore, The Lancet vol, 352 Aug. 1, 1998: 376. However, there are great complexities involved.

Despite the complexities, it would be greatly advantageous to employ somatic cell nuclear transfer (SCNT) technology to populate a mammalian egg (other than human), with a human nucleus in order to culture and expand stem cells without the associated controversy.

### SUMMARY OF THE INVENTION

The present invention is a method for the creation of stem cells substantially equivalent to the embryonic stem cells or embryonic germ cells without legal and ethical complications. The method avoids the controversy over the destruction of human life. The method is illustrated in the context of the creation of a totipoent cell equivalent to a human fertilized egg by total asexual process. This totipotent cell is a chimeric totipotent cell-contained total human genome. The present method uses somatic cell nuclear transfer (SCNT) technology by which the nucleus of an egg from a mammalian, other than human, was removed with care to avoid significant disruption of the egg plasma. Then a human somatic cell (any body cell other than germ cells) is arranged side by side with the de-nucleated egg. The de-nucleated egg and the human somatic cell are fused by chemical induced cell fusion. After the fusion, the newly formed chimeric cell is properly stimulated to start development (as is normally done during animal SCNT cloning). This chimeric cell culture continued with similar culture media used in culturing the fertilized egg for 4 to 5 days until the blastocyst is formed. At this stage, the blastocyst may be treated in one of two different ways. One way is to directly isolate the inner cell mass of the blastocyst and cultured as embryonic stem cells. The second choice is to implant this early blastocsyt into the uterus of a female animal with the identical species as the egg donor. After a proper period, the chimeric embryo is removed from the uterus and the embryonic germ cell is isolated from this chimeric embryo. In both ways, because the nuclei of these cells are identical to its human donor, the cells are substantially equivalent to the human embryonic stem cell. Also because it is originated by a human-animal chimeric cell, it is not suitable to carry the embryo forward.

Even with complete identical human genome, the pre-existing maternal signals in the egg are not human. At cellular level, this embryo could produce stem cells functionally equivalent to the normal human embryonic stem cells. However, on the organism level, under the influence of the pre-existing maternal messages, there would be a very high risk of physiological and morphological defects, and the organism most likely would not survive to the full term. Therefore, this asexually created chimeric totipotent cell would not and should not, and most likely could not, be carried beyond the blastocyst stage. Because the present invention does not involve the human sperm and egg, or the normal human fertilization process, and also does not involve the human egg as an essential component in the human cloning process, the process will generate only a totipotent cell but not a human fertilized egg. Also subsequently, the present invention will only generate puripotent cells equivalent to the human embryo stem cells but not a human embryo. Nevertheless, the method of the present invention will generate stem cells equivalent to the human embryonic stem cell or human embryonic germ cell without the controversy associated with the destruction of human embryos or the concern with human cloning.

### BRIEF DESCRIPTION OF THE DRAWINGS

Other objects, features, and advantages of the present invention will become more apparent from the following detailed description of the preferred embodiments and certain modifications thereof when taken together with the accompanying drawings in which:

FIG. 1 is a diagrammatical illustration of the present invention's process of creating chimeric stem cell that is substantially equivalent to the human embryonic stem cell or human embryonic germ cell.

FIG. 2 is a diagrammatical illustration of the alternative embodiment of the present invention's process of creating chimeric stem cell with less risk of defective result.

FIG. 3 is a diagrammatical illustration of the second alternative embodiment of the present invention's process of creating chimeric stem cell with less risk of defective result.

### DETAILED DESCRIPTION OF THE PREFERRED EMBODIMENTS

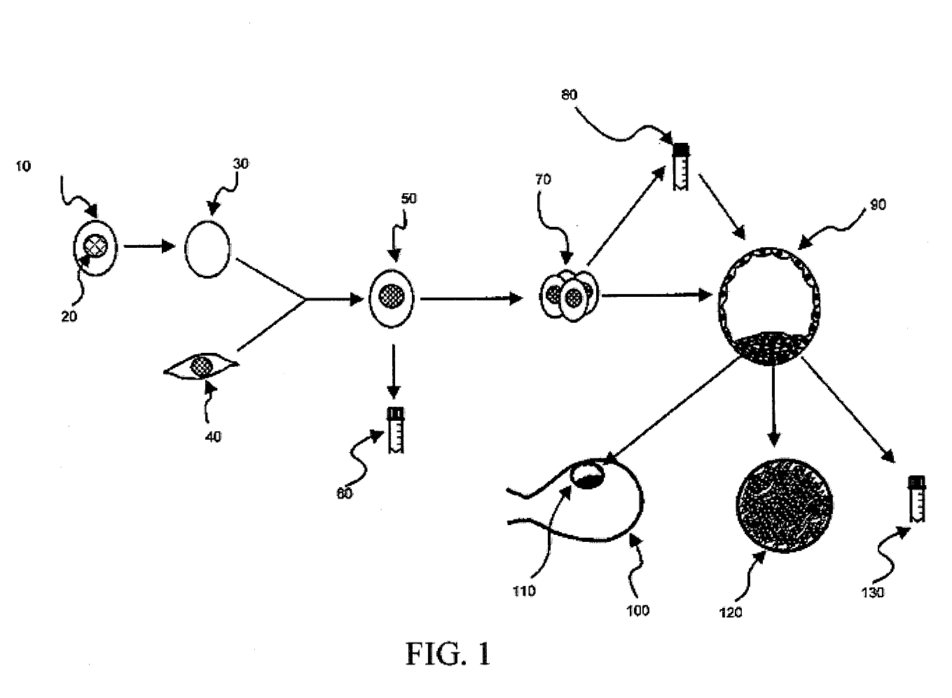

The present invention is a method for the asexual creation of chimeric embryo and production of stem cells equivalent to human embryonic stem cells without legal and ethical complications. The present method is illustrated diagrammatically in FIG. 1, which is a flow diagram showing the method in the context of creating a chimeric stem cell that is substantially equivalent to the human embryonic stem cell or human embryonic germ cell.

Initially, a fresh mature mammalian egg 10 (preferably from porcine) is harvested from the desired animal. The nucleus 20 of the egg is removed from the egg by micro-manipulator to form a de-nucleated egg 30. A human somatic cell 40 obtained from any part of the body, except germ cells, of the donor (preferably from the patient himself).

The human somatic cell may be a natural human somatic cell, or an artificial liposome having a cell nucleus isolated from a donor somatic cell, and mitochondria isolated from the cells of a nucleus donor. Alternatively, the human somatic cell may be an artificial cell obtained by transferring a cell nucleus from a donor's somatic cell, and mitochondria from the cells of a nucleus donor into a ghost cell created from an egg donor's or nucleus donor's red blood cell. A ghost cell is a cell with only cell membrane no any other component in the cell. Ghost cells are created by putting the red blood cells in a hypotonic solution (low salt so the water will continue to diffuse into the cells). The osmotic pressure bursts the cells and leaves the cell membrane as empty shells, with no contents. These cells can be chemically resealed by a well-known technique.

In any of the foregoing cases, the de-nucleated animal egg 30 and the human somatic cell 40 are then fused together by proper chemical treatment to create the chimeric cell 50. In accordance with the present invention, “mitochondria transfer” needs to be performed during this fusing process. Mitochondria are microorganisms that live symbiotically inside the most eukaryotic cells including all the mammalian cells except mature red blood cells. Mitochondria carry their own independent genetic material. However, the mitochondria will communicate with the cell nucleus on their control of gene expression. Mitochondria are highly specific among species. Thus, there is a large potential for incompatibility between the nucleus transferred from the human and the mitochondria of the animal egg. Therefore significant amount of human mitochondria, preferably the mitochondria from the nucleus donor, must be co-transferred with the nucleus to the de-nucleated animal egg in the preferred method of chimeric human stem cell cloning.

The chimeric cell can either be preserved by cryogenic technology as stock 60 for the future treatment or be activated to initiate cell division to form a small cluster of totipotent cells 70 by proper stimulation. The totipotent cells 70 can be either preserved by cryogenic technology as stock 80 for the future treatment or continuously cultured 4 to 5 days until the blastocyst 90 is formed. The blastocyst is preferably treated in one of three different processes. The first process is to isolate the inner cell mass from blastocyst and directly preserved by cryogenic technique to form the stem cell stock 130 that is equivalent to human embryo stem cell. The second process is to isolate the internal cell mass from the blastocyst 90 and directly transfer the cell to expansion culture 120 to supply current research and clinic applications. The third choice is to implant the blastocyst 90 back in the uterus 100 of the egg donor animal or the same species of the donor to allow the blastocyst 90 to continue to growth to 4˜5 weeks to form later stage blastocyst 110. The stem cells that are substantially equivalent to the human embryonic germ cell can be collected from the inner cell mass of the late stage blastocyst 90.

During the somatic cell nuclear transfer cloning process, nuclear reprogramming holds the key factor of the successful rate. Nuclear reprogramming is the process that resets all of the genes to an initial unmodified status. Normally, nuclear reprogramming occurs during the egg and sperm formation process. The newly formed fertilized egg is totipotent. It has ability to become any specific cell type involved in a body. However, organisms need specialized cells to perform many different functions during their development, and each type of cell must be programmed to only perform the designated function. Therefore, embryonic cells begin this differentiation process. In different steps during the differentiation, certain genes which have nothing to do with the targeted future-function will be shut down. This shutting-down process usually occurs by chemical modification of the nucleic acid in certain positions. One known chemical modification method is methylation on the nucleotide bases. However, there are other ways, and the various processes are not well-understood by the scientists.

In light of the above, the nuclear reprogramming is necessary to remove all these barriers to recover the full potential of the entire genetic material—DNA. Reprogramming was not a proper name by definition scientifically, because the word of reprogramming is not necessary interpreted as reset the function to ground zero. It just commonly used by the researchers, it is more like reverse the programming process.

The higher incomplete reprogramming will reflect on higher failure rate due to genetic dysfunction. Therefore, recycling the totipotent cell of the first cycle through another step of nuclear reprogramming may significantly reduces the incomplete nuclear reprogramming. The cycle can be repeated more than once, it may vary among the source of egg donors.

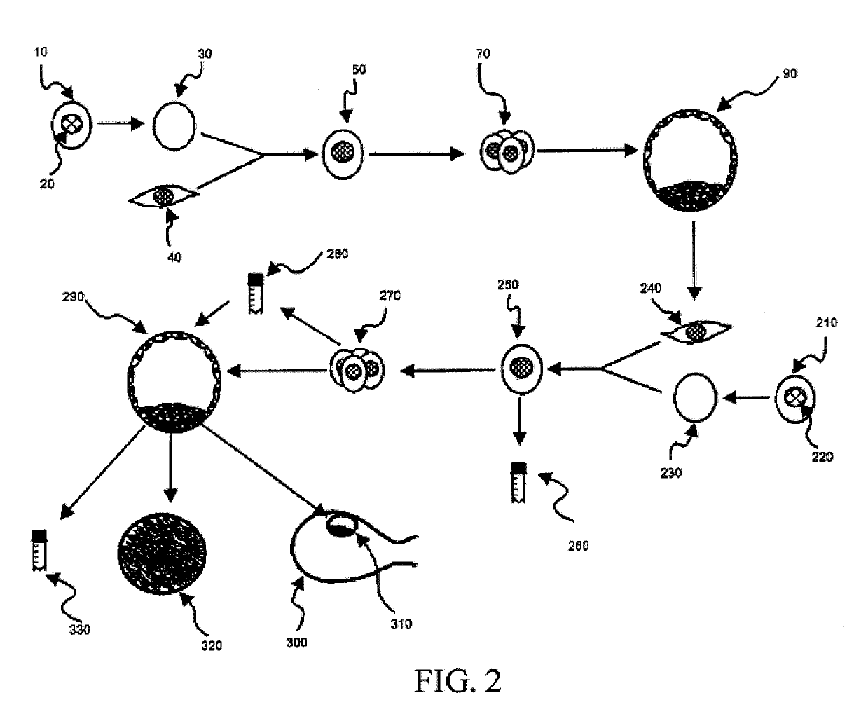

An alternative embodiment of the present method of generating stem cells equivalent to human embryo stem cells is illustrated in FIG. 2. Initially, the process is the same as the illustrated in FIG. 1. However, when the blastocyst 90 is developed, the inner cell mass is isolated from the blastocyst 90. One of these isolated cells 240 will be used as the somatic cell to be fused with another de-nucleated animal egg 230 prepared by removing the nucleus 220 from the animal egg 210, preferably from the same origin as egg 10 or the same species. The development process is repeated as the first cycle until a new blastocyst 290 is developed. During the developmental process the cells can be preserved at various stages. At the initial stage the chimeric cell 250 (equivalent to fertilized egg) and the totipotent cells 270 developed by initial several rounds of cell division can all be preserved by cryogenic process. From the blastocyst 290 the human embryonic stem cells equivalent chimeric stem cell will be isolated from the inner cell mass of the blastocyst 290 generated by the second cycle. These pluripotent cells can be either preserved by cryogenic process 280 for future application or immediately expanded by proper laboratory culture process 320 for research or clinical applications. The blastocyst 290 can also be implanted into the donor animal's or foster mother's uterus 300 (preferably the same species as the egg donor), and this is followed by allowing 4˜5 days of further development from blastocyst 290 to blastocyst 310. Afterward, the human embryonic germ cell equivalent stem cell can be isolated from the inner cell mass of blastocyst 310.

During the embryo development process, differentiation starts as early as the embryo development proceeding from the cluster of totipotent cells into blastocyst of pluripotent cells. This means that certain limitations must be applied to the pluripotent cells. For example, methyl groups were added to some of the nucleotides of the genes that became useless or inappropriate for the newly differentiated cell type to prevent the expression of these gene products. The idea of re-cycle cloning is to provide more opportunity to remove the blockage of the genes due to the differentiation process, if the first cycle of reprogramming was incomplete. Starting the re-cycle cloning before the blastocyst formation can avoid the new restriction being added onto the genome. Therefore starting the re-cycle cloning process from the previous cycle's cluster of total potent cell can provide a better opportunity for complete nuclear reprogramming.

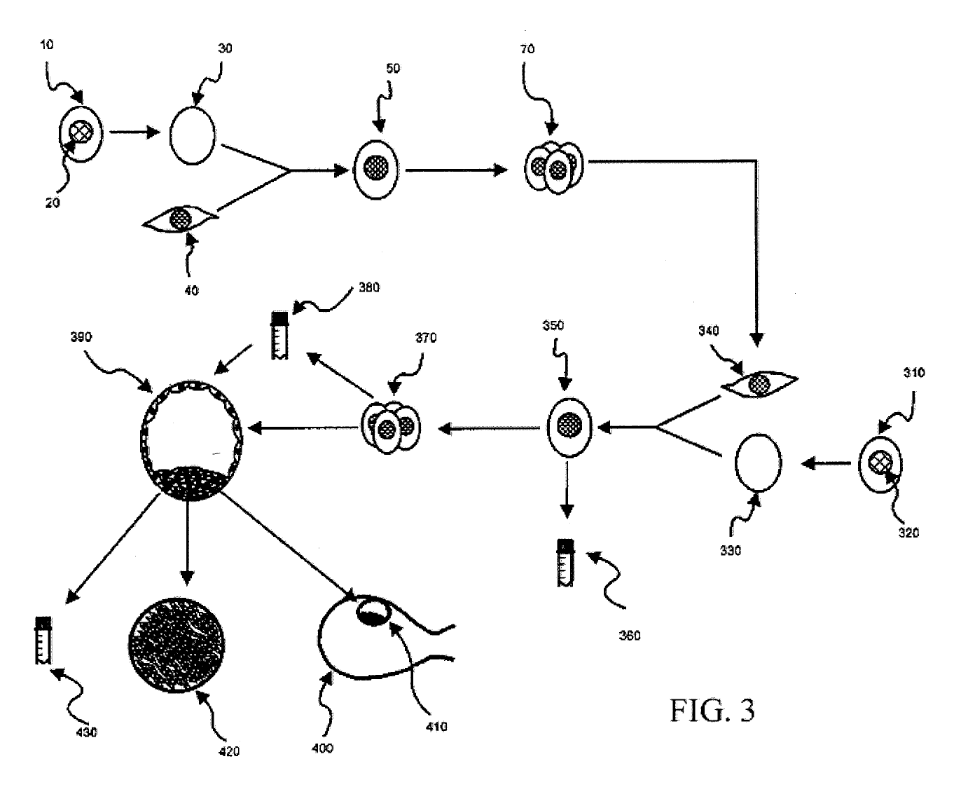

A third preferred alternative embodiment based on the same mechanism of the process of generating stem cells equivalent to human embryo stem cell is illustrated in FIG. 3. Initially, the process is the same as the illustrated in FIGS. 1 and 2. Before the blastocyst is developed, totipoent cells are isolated from the cluster of the totipotent cells 70. One of these isolated cells 340 will be used as the somatic cell to be fused with another de-nucleated animal egg 330 that is created by removing the nucleus 320 from the animal egg 310 preferably from the same origin as egg 10 or the same species. The optimal number of the cycle of re-cloning shall be determined on species bases. Following the last cycle of nucleus transfer, the clone will be cultured to form the cluster of totipotent cells 370. The totipotent cell is then harvested and preserved in container 380 by known cryogenic techniques or allowing them to continue develop until a new blastocyst 390 is formed. During the developmental process the cells can be preserved at various stages. From the blastocyst 390 the human embryonic stem cells equivalent chimeric stem cells will be isolated from the inner cell mass. These pluripotent cells can be either preserved by cryogenic process 430 for future application or immediately expanded by proper laboratory culture process 420 for research or clinical applications. The blastocyst 390 can also be implanted into the donor animal's or foster mother's uterus 400 (preferably the same species as the egg donor), followed by allowing 4˜5 days of further development from blastocyst 390 to blastocyst 410. Then the human embryonic germ cells equivalent stem cells can be isolated from the inner cell mass of blastocyst 310.

The foregoing embodiments of the present method are not limited to clone cells equivalent to human embryo stem cells. The invention is broadly usable for cloning stem cell for any species, including endanger species, and in other medical and veterinary applications. The application field of this invention includes, but is not necessarily limited to, medical, pharmaceutical, molecular biology and veterinary medicine.

Having now fully set forth the preferred embodiment and certain modifications of the concept underlying the present invention, various other embodiments as well as certain variations and modifications of the embodiments herein shown and described will obviously occur to those skilled in the art upon becoming familiar with said underlying concept. For example, while the invention has been disclosed with reference to generating stem cells equivalent to human embryonic stem cells and human embryonic germ cells without creating and destroying natural human embryos for medical researches and clinical applications, those skilled in the art will recognize that the invention will be useful for any procedure requiring the creating embryonic pluripotent cells. It is to be understood, therefore, that the invention may be practiced otherwise than as specifically set forth in the appended claims.

I claim:

1. A method for the asexual creation of chimeric embryo, comprising the steps of:
harvesting a mature mammalian egg from a non-human animal;
removing the nucleus of the egg to form a de-nucleated egg;
providing a human somatic cell from any part of the body, except germ cells, of a donor;
fusing the de-nucleated animal egg and the human somatic cell together to create a chimeric cell;
transferring mitochondria from the nucleus donor with the nucleus to the de-nucleated animal egg.

2. The method according to claim 1, further comprising the step of preserving said chimeric cell by cryogenic technology.

3. The method according to claim 1, further comprising the step of activating said chimeric cell to initiate cell division to form a small cluster of totipotent cells.

4. The method according to claim 3, further comprising the step of preserving said totipotent cells by cryogenic technology.

5. The method according to claim 3, further comprising the step of culturing said totipotent cells until a blastocyst is formed.

6. The method according to claim 5, further comprising the step of isolating inner cell mass from the blastocyst to form an isolated cell, and fusing said isolated cell with another de-nucleated animal egg.

7. The method according to claim 1, wherein said step of providing a human somatic cell from any part of the body further comprises providing a human somatic cell comprising any one from among the group of:
a) a natural human somatic cell;
b) an artificial liposome having a cell nucleus isolated from a donor somatic cell and mitochondria from the cells of a nucleus donor;
c) an artificial cell obtained by transfering a cell nucleus from a donor's somatic cell and mitochondria from the cells of a nucleus donor into a ghost cell created from an egg donor's or nucleus donor's red blood cell.
